(19) United States
(12) Patent Application Publication
(10) Pub. No.: US 2008/0275370 A1
(43) Pub. Date: Nov. 6, 2008

(54) APPARATUS AND METHOD FOR TREATING VISUAL DISORDERS

(76) Inventors: Jon T. McIntyre, Newton, MA (US); Isaac Ostrovsky, Wellesley, MA (US); Victor Shukhat, Canton, MA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/742,644

(22) Filed: May 1, 2007

(51) Int. Cl. A61N 7/00

(52) U.S. Cl. 601/2

(57) ABSTRACT

In one embodiment, an apparatus includes an elongate body having a proximal end and a distal end and an ultrasound transducer coupled to the distal end of the elongate body. A cooling member is coupled to the ultrasound transducer. The cooling member and/or the ultrasound transducer is configured to directly contact an outer surface of an eye such that ultrasound energy can be applied to a targeted portion of the eye. A method includes disposing a medical device such that a distal end of the medical device is in contact with an outer surface of an eye. The medical device includes an elongate body and an ultrasound transducer coupled to a distal end of the elongate body. Ultrasound energy is applied to a targeted portion of the eye while the medical device is disposed in contact with the outer surface of the eye to reshape the cornea of the eye.

### FIELD OF THE INVENTION

The disclosed invention relates generally to a medical device and more particularly to a medical device configured to treat various visual disorders.

### BACKGROUND

A center portion of the cornea of an eye (in the field of vision) possesses a radius of curvature less than the curvature of the remaining portion of the eye. The cornea and the lens cooperatively focus light entering the eye onto the retina. Various visual disorders, such as, hyperopia, myopia and keratoconus, can hinder this process by causing the focal point of light to occur at a location other than the retina. One method of treating such disorders is to reshape the cornea of the eye. For example, hyperopia can be corrected by increasing the curvature of the cornea to decrease the dioptric power. To treat or correct myopia and keratoconus, the cornea is flattened.

Various known methods have been employed to treat the above-mentioned conditions, including surgical procedures such as radial keratomy (including LASIK), and thermal methods of treatment including the use of heated rods, radiofrequency devices and/or microwaves. Often, other devices are needed to perform such a procedure, such as a device to provide a coolant to the treatment site during the procedure.

Problems with such methods of treatment can include, for example, risks associated with invasive surgery, damage created by the thermal element, and the inability to control or direct the treatment to the targeted treatment site.

Thus, there is a need for an apparatus and method for treating various visual disorders that provides improved control and direction of treatment, and cooling capabilities to reduce thermal effects.

### SUMMARY OF THE INVENTION

In one embodiment, an apparatus includes an elongate body having a proximal end and a distal end and an ultrasound transducer coupled to the distal end of the elongate body. A cooling member is coupled to the ultrasound transducer. The cooling member and/or the ultrasound transducer is configured to directly contact an outer surface of an eye such that ultrasound energy can be applied to a targeted portion of the eye. A method includes disposing a medical device such that a distal end of the medical device is in contact with an outer surface of an eye. The medical device includes an elongate body and an ultrasound transducer coupled to a distal end of the elongate body. Ultrasound energy is applied to a targeted portion of the eye while the medical device is disposed in contact with the outer surface of the eye to reshape the cornea of the eye.

### BRIEF DESCRIPTION OF THE DRAWINGS

FIG. 1 is a schematic illustration of a medical device according to an embodiment of the invention.

FIG. 2 is a side view of an eye illustrating an example of a normal curvature of a cornea.

FIG. 3 is a side view of an eye illustrating an example of a flattened cornea.

FIG. 4 is a side view of an eye illustrating an example of an increased curvature of a cornea.

FIG. 5 is a side view of a portion of a medical device according to an embodiment of the invention shown positioned adjacent to an eye.

FIG. 6 is a distal end view of the medical device of FIG. 5 taken along line 6-6 in FIG. 5.

FIG. 7 is a side cross-sectional view of the medical device of FIG. 5 shown positioned adjacent to an eye.

FIG. 8 is a side view of a portion of a medical device according to another embodiment of the invention.

FIG. 9 is a distal end view of the medical device of FIG. 8 taken along line 9-9 in FIG. 8.

FIG. 10 is a side cross-sectional view of the medical device of FIG. 8 shown adjacent an eye.

FIG. 11 is a side cross-sectional view of a medical device according to another embodiment of the invention.

FIG. 12 is a distal end view of the medical device of FIG. 11.

FIG. 13 is a side cross-sectional view of a medical device according to another embodiment of the invention.

FIG. 14 is a flowchart illustrating a method according to an embodiment of the invention.

### DETAILED DESCRIPTION

The apparatuses and methods described herein can be used to treat various visual disorders associated with an eye, such as hyperopia, myopia and keratoconus. The medical devices include the use of targeted ultrasonic energy to, for example, reshape the cornea of an eye. One or more ultrasound transducers can be placed directly in contact with an outer surface of an eye for optimal tissue coupling with the ultrasound transducer. A cooling member can also be provided to reduce undesirable thermal effects to the surface of the eye.

In one embodiment, an apparatus includes an elongate body having a proximal end and a distal end and an ultrasound transducer coupled to the distal end of the elongate body. A cooling member is coupled to the ultrasound transducer. The cooling member and/or the ultrasound transducer is configured to directly contact an outer surface of an eye such that ultrasonic energy can be applied to a targeted portion of the eye.

A method of using a medical device as described herein includes disposing a medical device such that a distal end of the medical device is in contact with an outer surface of an eye. The medical device includes an elongate body and an ultrasound transducer coupled to a distal end of the elongate body. Ultrasound energy is applied to a targeted portion of the eye while the medical device is disposed in contact with the outer surface of the eye to reshape the cornea of the eye.

In another embodiment, an apparatus includes an elongate body having a proximal end and a distal end and defining a lumen couplable to a source of suction. An ultrasound transducer is coupled to the distal end of the elongate body that is configured to contact an outer surface of an eye such that ultrasound energy can be applied to a targeted portion of the eye.

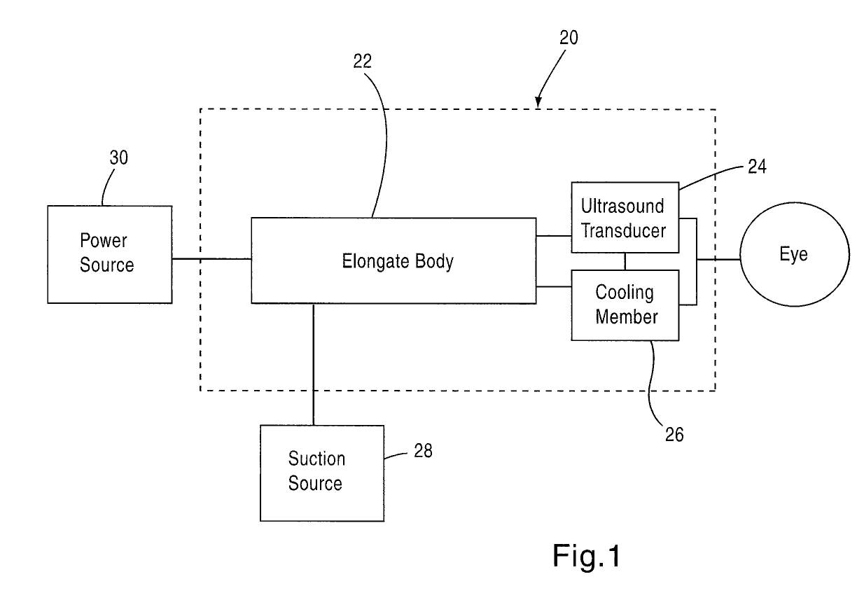

FIG. 1 is a schematic illustration of a medical device (also referred to herein as an “apparatus”) according to an embodiment of the invention. A medical device 20 includes an elongate body 22 and one or more ultrasound transducers 24 coupled to a distal end of the elongate body 22. A cooling member 26 can optionally be coupled to the ultrasound transducer 24 and/or the elongate body 22, at a distal end thereof.

The ultrasound transducer(s) 24 can be formed in various shapes and sizes, and can be formed, for example, with a piezoelectric crystal, such as with PZT-8. In alternative embodiments, a piezoelectric film can be used as the ultrasound transducer. In some embodiments, the ultrasound transducer 24 is a crystal formed as an annular ring defining a center opening. In some embodiments, multiple ultrasound transducers 24 are arranged in a circular or ring pattern as described below with reference to specific embodiments. Such a ring or circular shape allows for delivery of ultrasonic energy around the pupil of the eye, outside of the field of vision. The ultrasound transducer(s) 24 is configured to be placed directly on the surface of an eye for optimal tissue coupling. In an embodiment including a cooling member 26, the cooling member 26 is configured to be placed directly on the surface of the eye as will be described in more detail below. The ultrasound transducer(s) 24 can be used, for example, to reshape a cornea of the eye to treat various eye disorders.

The elongate body 22 can define a lumen (not shown in FIG. 1) that can be coupled to a source of suction 28. The lumen can be defined longitudinally through a center of the elongate body 22 and a center of the ultrasound transducer 24. Alternatively, the lumen of the elongate body 22 can be disposed annularly around the ultrasound transducer 24, as will be described in more detail below with reference to specific embodiments. The location of the applied suction (e.g., the location of the lumen of the elongate body 22) may depend on the particular disorder being treated (e.g., near-sightedness or far-sightedness), so as not to affect the reshaping of the cornea in an undesired fashion.

The elongate body 22 can also define a passageway (not shown in FIG. 1) extending along a length of the elongate body 22 and having a distal end terminating at the ultrasound transducer 24. The passageway provides a means for the containment and/or circulation of fluid (e.g., coolant, water) behind the ultrasound transducer 24. This allows the ultrasound transducer 24 to remain cool while being placed directly on a surface of an eye and without creating undesirable thermal effects on the surface of the eye. This can also eliminate the need for the application of a coolant to the surface of the eye, such as saline, during treatment of the eye. In some embodiments, the elongate body 22 defines a dual channels within the passageway to allow for fluid to circulate within the passageway. For example, fluid can flow distally toward the ultrasound transducer(s) 24 through one channel and then flow back proximally through the other channel.

In some embodiments, where the cycle time (e.g., the cycle time of the application of the ultrasound transducer 24 in contact with the eye) is very short, the medical device 20 may not include a fluid passageway. In such an en embodiment, because there is a short cycle time, coolant may not be needed, as the heat generated by the ultrasound transducer(s) 24 are minimal.

The cooling member 26 is configured to be coupled to a distal end of the ultrasound transducer 24 and directly contact the surface of an eye. For example, the cooling member 26 can be adhesively coupled to the elongate body 22. The cooling member 26 defines an interior chamber (not shown in FIG. 1) configured to receive and/or contain a fluid (e.g., saline or water) for energy coupling between the ultrasound transducer 24 and the surface of the eye. For example, in some embodiments, the cooling member 26 has fluid disposed within a closed interior chamber. In other embodiments, the cooling member 26 is configured such that fluid can flow through the interior chamber as described above for the fluid circulation within the passageways of the elongate body 22. In some embodiments, the cooling member 26 is formed with a translucent material. The cooling member 26 can be formed, for example with polyethylene terephthalate (PET) or polyethylene (PE), or ethylene vinyl acetate (EVA). Other suitable materials can be used, such as, for example, materials that are thin relative to the ultrasound energy wavelength so as not to reflect the energy and have an acoustic impedance similar to that of water so as to transmit the ultrasound energy. The cooling member 26 can assist in cooling the ultrasound transducer 24, and help manage the temperature of the surface of the eye during treatment. The cooling member can also be in the form of a compliant “pillow” having thin walls that can conform to the contour of the eye and assist in maintaining the medical device 20 in contact with the surface of the eye.

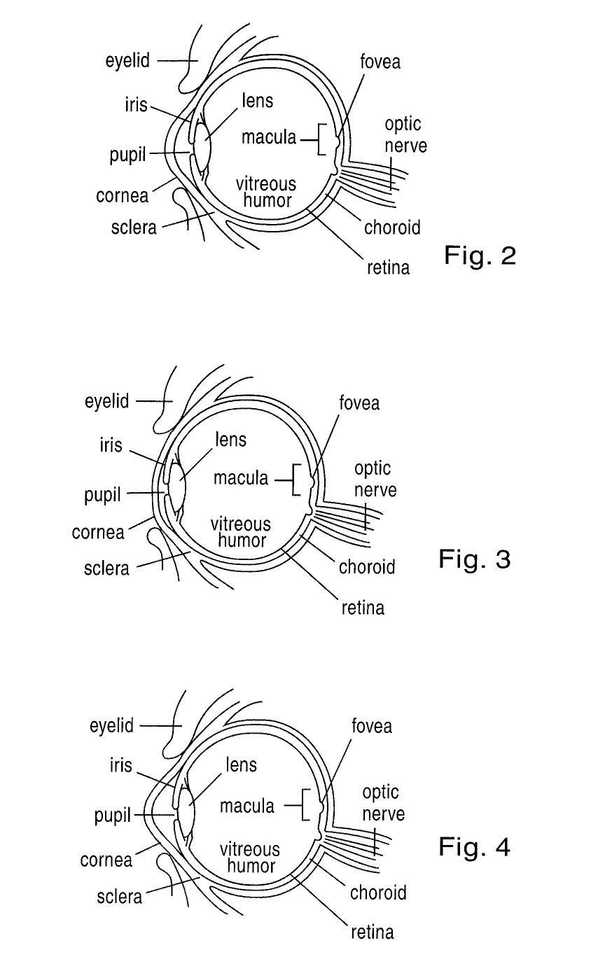

In use, a distal end of the medical device 20 can be placed directly in contact with a surface of an eye. Depending on the particular embodiment, either the ultrasound transducer 24 or the cooling member 26, if the device 20 includes a cooling member, will directly contact the outer surface of the eye. In an embodiment in which suction is provided, a gentle suction can be applied through the lumen of the elongate body 22 to the surface of the eye to stabilize the position of the medical device 20 during treatment. For example, suction or vacuum can be provided in a range between atmospheric pressure and minus 1 atm. Suction can also be applied simultaneously to ultrasound energy delivery in order to aid in reshaping the cornea. The medical device 20 can be coupled to a power source 30, such as a standard radio frequency (RF) generator, to energize the ultrasound transducer 24. For example, the ultrasound transducer(s) 24 can be coupled to the power source 30 via a wire. Ultrasonic energy can then be applied to a selected or targeted treatment site on the eye to, for example, reshape a tissue, such as the cornea, of the eye. For example, as shown in FIG. 3, a cornea can be reshaped so as to be less curved (e.g., more flattened) than a normal cornea (shown in FIG. 2) to treat for example, myopia and keratoconus. In other cases, the cornea can be reshaped so as to have more curvature than a normal cornea, such as the cornea shown in FIG. 4, to treat, for example, hyperopia.

The ultrasound can have a frequency range, for example, for shear mode of 2-20 MHz, and a frequency range for a traditional piston mode of 20-100 MHz. The treatment time can be in the range of, for example, 1-5 seconds per eye. A goal of the treatment can be to increase the temperature of the collagen to a temperature greater than, for example, 42 degrees Celsius. In some embodiments, a camera, such as an infrared camera, or other viewing device can be disposed through the lumen of the elongate body to monitor the temperature of the eye. The treatment frequency can be in the range, for example, of 2.5 MHz to 100 MHz. The medical device 20 can be used, for example, in a shear mode of resonance or thickness mode. The shear mode will allow for the use of lower frequencies without deep penetration. The suction provided via the lumen of the elongate body 22 can also assist in reshaping the tissue by applying an appropriate tension to the surface of the eye. Thus, the ultrasound energy and/or the suction force can be used to reshape a selected tissue area of the eye.

Having described above various general principles, several exemplary embodiments of these concepts are now described. These embodiments are only examples, and many other configurations of medical device 20 and its various components are contemplated by the principles of the invention, and will be apparent to the artisan in view of the general principles described above and the exemplary embodiments.

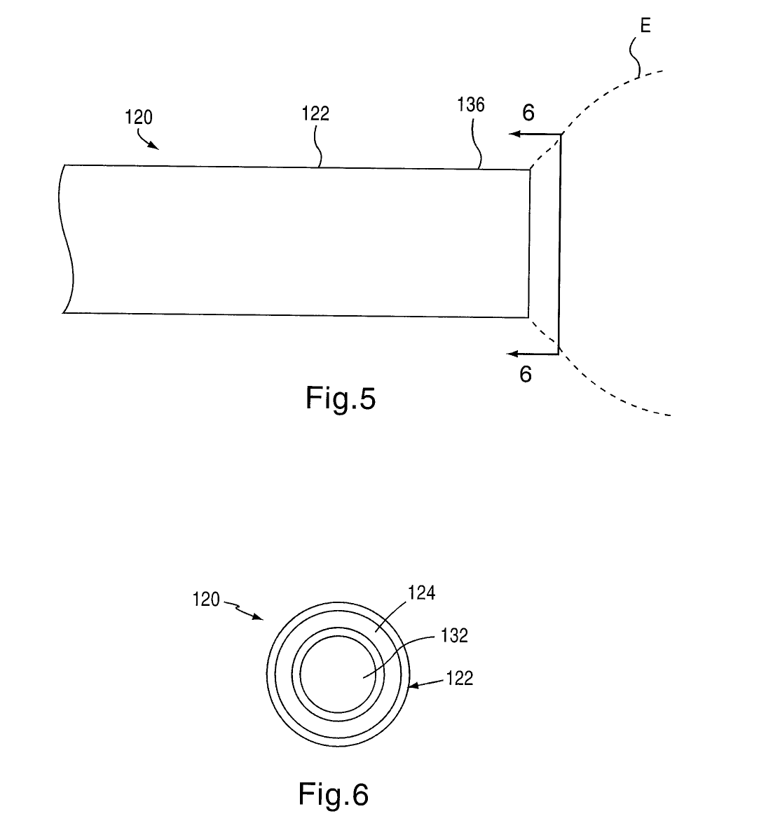

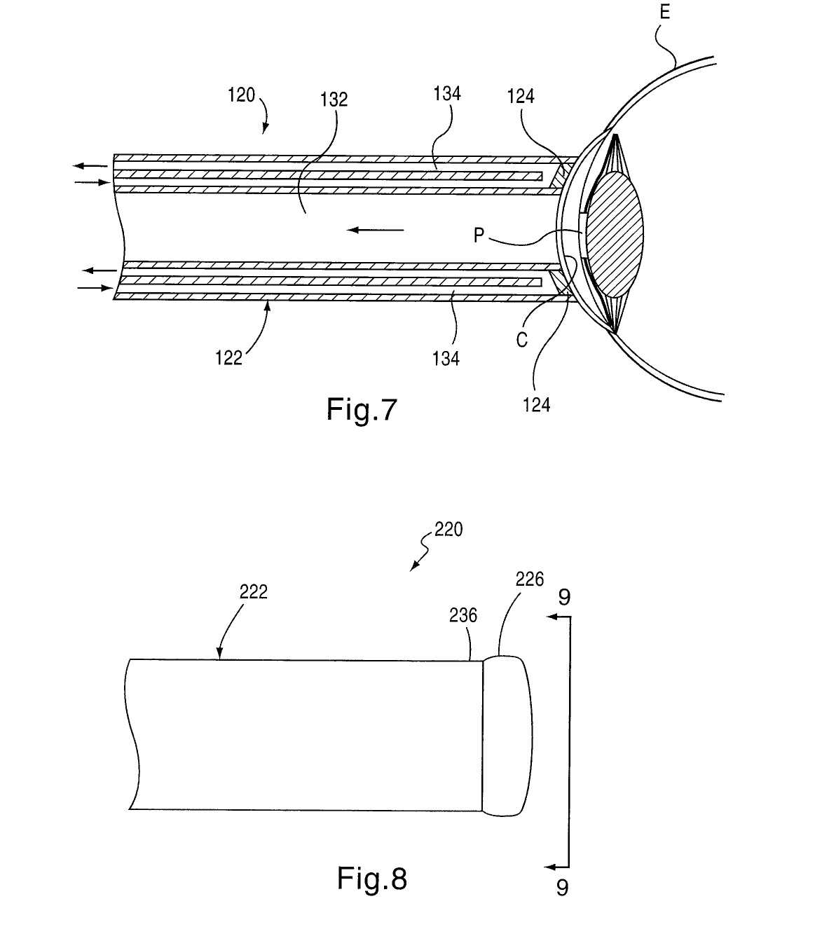

FIGS. 5-7 illustrate a medical device according to an embodiment of the invention. A medical device 120 includes an elongate body 122 and an ultrasound transducer 124. In this embodiment, the elongate body 122 defines a lumen 132 and an annular passageway 134 disposed proximally of the ultrasound transducer 124, as shown in FIGS. 6 and 7. The lumen 132 can be coupled to a suction source (not shown) and the passageway 134 can be coupled to a source of fluid (not shown). In this embodiment, the passageway 134 includes dual channels, which allows the fluid to be circulated through the passageway 134. For example, fluid can be provided to the passageway 134 from a fluid source (not shown) using, for example, a pumping device (not shown) coupled to the medical device 120. The ultrasound transducer 124 is coupled to a power source (not shown) via, for example a wire, to energize the ultrasound transducer 124.

In this embodiment, the ultrasound transducer 124 is a single crystal in the form of an annular ring. The ultrasound transducer 124 includes a distal end having a contoured shape configured to directly contact a surface of an eye E, as shown in FIG. 7. The fluid circulating within the passageway 134 can cool the ultrasound transducer 124 during use.

For use in the treatment of the eye E, a distal end 136 of the medical device 120 can be positioned such that the ultrasound transducer 124 directly contacts an outer surface of the eye E outside of the field of vision and the pupil P, as shown in FIG. 7. Suction can be provided through the lumen 132 to stabilize the position of the medical device 120 relative to the eye E. With fluid circulating in passageway 134 cooling the ultrasound transducer 124, ultrasonic energy can be applied to raise the temperature of the collagen and reshape the cornea C of the eye E. In some cases, the suction force may also be used to assist in reshaping the tissue (e.g., cornea) of the eye.

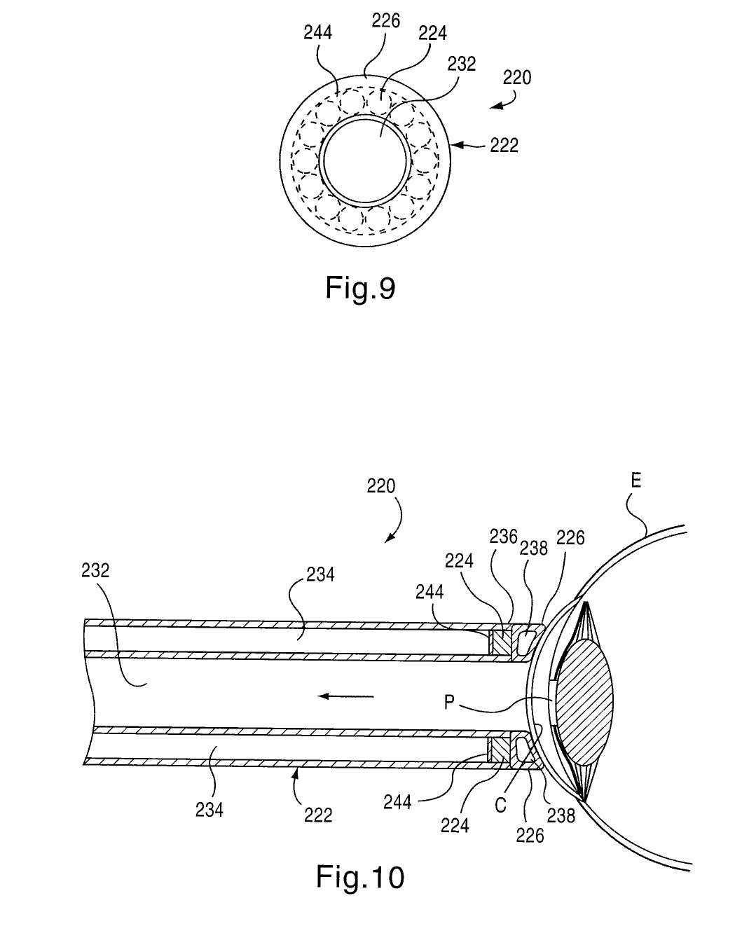

FIGS. 8-10 illustrate a medical device according to another embodiment of the invention. A medical device 220 includes an elongate body 222, multiple ultrasound transducers 224 (FIGS. 9 and 10), and a cooling member 226. As with the previous embodiment, the elongate body 222 defines a lumen 232 (FIGS. 9 and 10) that can be coupled to a source of suction (not shown). The suction can provide the same functions as previously described. The elongate body 222 also defines an annular passageway 234 (FIG. 10) configured to receive fluid therein to cool the ultrasound transducers 224 during use. In this embodiment, the passageway 234 is shown without dual channels as in the previous embodiment, although it should be understood that a dual channel passageway can alternatively be used to provide for circulating fluid.

In this embodiment, the ultrasound transducers 224 are crystals configured in a circle or ring, as shown in FIG. 9. The ultrasound transducers 224 are coupled to a sheet 244 formed, for example, with stainless steel. The thickness of this sheet, can be, for example, equal to 2N×¼ (where N is a whole number) of the wavelength to make sure that ultrasound energy coupled to the sheet can get reflected back into the transducer and into the eye. The thickness can also be equal to (2N+1)× 1/4 of the wavelength. In such a case, the ultrasound energy can pass through the sheet into fluid behind the sheet.

If the sheet is made such that there is an opening behind the transducer, for example, such as a ring, the back of the transducer can be exposed to air. In this case, the transducer can work in a more efficient manner since the energy generated on the back of the transducer would be reflected into the eye. A source of power (not shown) can be coupled to the ultrasound transducers 224, for example, via a wire coupled to the sheet 244. The sheet 224 and/or the ultrasound transducers 224 can be coupled to the distal end 236 of the elongate body 222 with, for example, an adhesive.

The cooling member 226 can also be coupled to the distal end 236 of the elongate body 222 with, for example, and adhesive. The cooling member 226 defines a chamber 238 that can receive and hold fluid to help cool the ultrasound transducers 224 during use and to provide energy coupling between the ultrasound transducers 224 and the tissue of the eye. In some embodiments, a medical device can be configured such that fluid is circulated within the chamber 238 in a similar manner as with the passageway 134 of medical device 120. The cooling member 226 can be formed with, for example, a thin layer of translucent material, such as a translucent PET or PE, that is flexible and/or pliable to allow the cooling member 226 to conform to a surface of an eye E, as shown in FIG. 10. Such a configuration assist in maintaining the medical device 220 in contact with the surface of the eye during a medical procedure.

The medical device 220 can be used in the same manner as described above for the previous embodiment. As shown in FIG. 10, the medical device 220 can be disposed adjacent an eye E such that the cooling member 238 directly contacts an outer surface of the eye E and surrounds the pupil P. Ultrasonic energy can be applied to reshape a tissue, such as a cornea C of the eye E. Suction can be applied as previously described to maintain the position of the medical device 220 and/or to assist in reshaping the tissue of the eye.

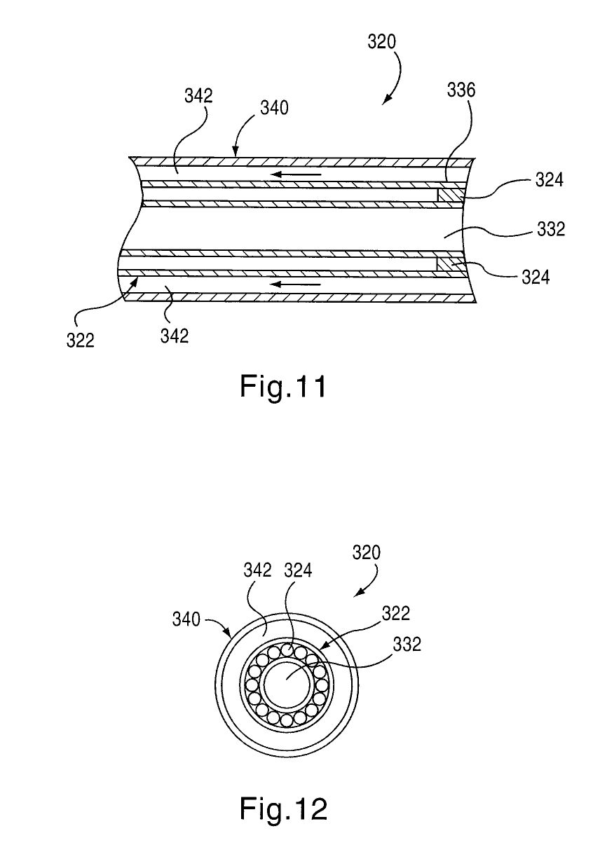

FIGS. 11 and 12 illustrate a medical device according to another embodiment of the invention in which suction is provided through an annular lumen. In this embodiment, a medical device 320 includes a first elongate body 322 and a second elongate body 340. The first elongate body 322 is disposed within the second elongate body 340 such that an annular lumen 342 is defined between the first elongate body 322 and the second elongate body 340. The lumen 342 can be coupled to a source of suction (not shown) to provide suction to stabilize the position of the medical device 320 and/or help reshape a tissue of an eye during a procedure on an eye as previously described.

Multiple ultrasound transducers 324 in the form of ultrasound crystals are coupled to a distal end 336 of the first elongate body 322. The ultrasound transducers 324 can include a contoured surface to conform to an outer surface of an eye. Alternatively, the medical device 320 can include a single ultrasound transducer as in medical device 120 previously described. Also, although not shown, the medical device 320 can also include a cooling member, such as cooling member 226. The first elongate body 322 also defines a lumen 332 that can optionally be coupled to a suction source to provide additional suction to the medical device 320, or can be coupled to a source of fluid, such as saline, and be used to irrigate the surface of the eye. In some embodiments, the first elongate body 322 does not define a lumen.

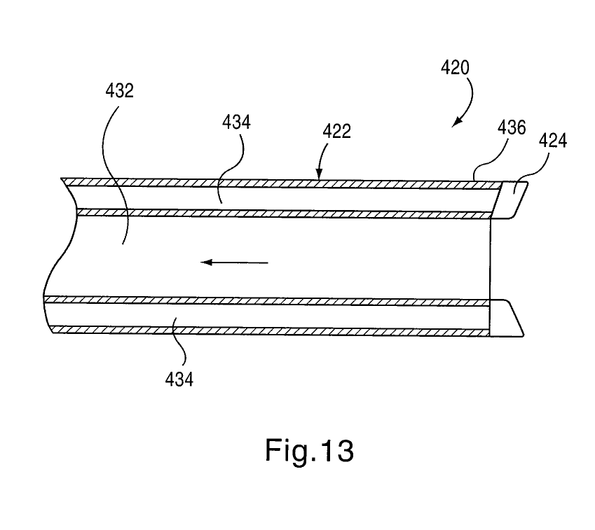

FIG. 13 illustrates yet another embodiment of a medical device. A medical device 420 includes an elongate body 422 and an ultrasound transducer 424 coupled at a distal end 436 of the elongate body 422. The elongate body 422 defines a lumen 432 and an annular passageway 434 disposed proximally of the ultrasound transducer 424. As with the previous embodiments, the lumen 432 can be coupled to a suction source (not shown) and the passageway 434 can be coupled to a source of fluid (not shown). The ultrasound transducer 424 is coupled to a power source (not shown) via, for example a wire, to energize the ultrasound transducer 424. In this embodiment, the ultrasound transducer 424 is shown as a single crystal in the form of an annular ring, but alternatively can include multiple crystals as described above. Also in this embodiment, the ultrasound transducer 424 is coupled to the distal end 436, without being enclosed within the walls of the elongate body 422 as shown in the previous embodiments.

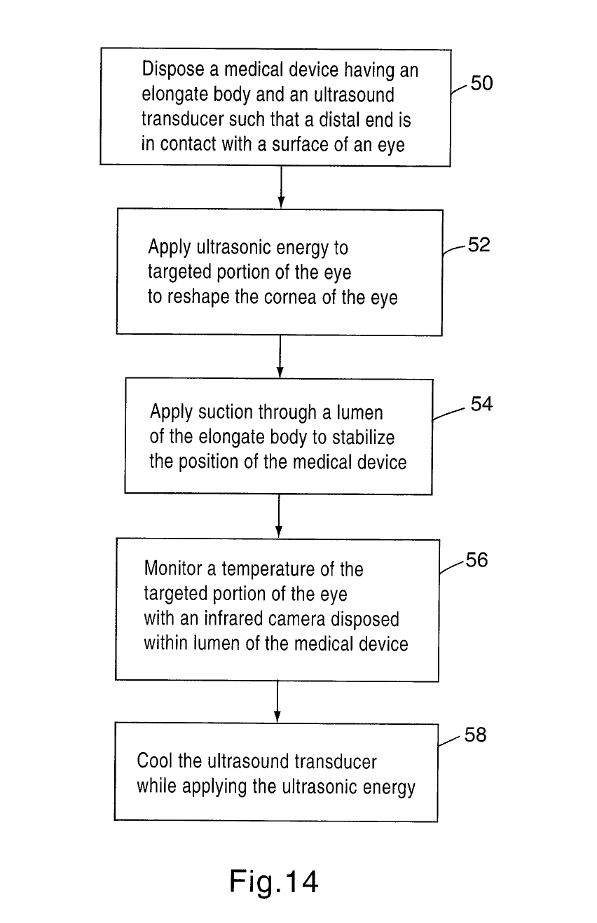

FIG. 14 is a flowchart illustrating a method according to an embodiment of the invention. A method includes at 50, disposing a medical device such that a distal end of the medical device is in contact with a surface of an eye. The medical device includes an elongate body and an ultrasound transducer coupled to a distal end of the elongate body. The medical device can include any of the embodiments of a medical device described herein or any combination of the various features of a medical device described herein. After disposing the medical device such that the distal end is in contact with the surface of the eye, ultrasonic energy is applied to a targeted portion of the eye to reshape the cornea of the eye at 52. For example, the cornea can be reshaped by increasing the curvature of the cornea. In another example, the cornea can be reshaped by decreasing the curvature of the cornea.

Also after disposing the medical device in contact with the eye, suction can be applied through a lumen of the elongate body to help stabilize the position of the medical device relative to the eye, and can in some cases assist in reshaping the cornea, at 54. The suction can be applied simultaneously or sequentially with the application of ultrasound energy. At 56, a temperature of the targeted portion of the eye can be monitored during the treatment with ultrasonic energy with, for example, an infrared camera disposed within a lumen of the elongate body. Also during the treatment with the ultrasonic energy, at 58 the ultrasound transducer can be cooled with a cooling member coupled to the ultrasound transducer. Alternatively, pressure can be applied through another lumen to further reshape the cornea.

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

The previous description of the embodiments is provided to enable any person skilled in the art to make or use the invention. While the invention has been particularly shown and described with reference to embodiments thereof, it will be understood by those skilled in art that various changes in form and details may be made therein without departing from the spirit and scope of the invention.

For example, a medical device 20 (120, 220, 320, 420) can include any combination or sub-combination of the various features and components described herein. Thus, a medical device 20 (120. 220, 320, 420) can include other configurations, shapes and materials not specifically illustrated, while still remaining within the scope of the invention. For example, a medical device can include one or more ultrasound transducers in various configurations. A medical device can include a cooling member on a distal side of the ultrasound transducer(s), a passageway with fluid on a proximal side of the ultrasound transducer(s), or both for cooling the ultrasound transducer(s) during use, regardless of the particular shape and configuration of the ultrasound transducer(s). In addition, one or more lumens can be provided that can be coupled to a source of suction and/or for providing irrigation. In another example, the ultrasound transducer(s) can be coupled to the elongate body at various positions relative to the elongate body. For example, in any of the embodiments, the ultrasound transducer(s) can be coupled to a distal end of the elongate body such that it is enclosed at least partially within walls of the elongate body or disposed on a distal most end of the elongate body as illustrated, for example, in FIG. 10.

What is claimed is:

1. An apparatus, comprising:
an elongate body having a proximal end and a distal end;
an ultrasound transducer coupled to the distal end of the elongate body; and
a cooling member coupled to the ultrasound transducer,
at least one of the cooling member or the ultrasound transducer configured to directly contact an outer surface of an eye such that ultrasound energy can be applied to a targeted portion of the eye.

2. The apparatus of claim 1, wherein the elongate body defines a lumen couplable to a source of suction.

3. The apparatus of claim 1, wherein the ultrasound transducer is formed as an annular ring around a lumen defined by the elongate body.

4. The apparatus of claim 1, wherein the cooling member is coupled to a distal end of the ultrasound transducer.

5. The apparatus of claim 1, wherein the cooling member is coupled to a proximal end of the ultrasound transducer.

6. The apparatus of claim 1, wherein the ultrasound transducer includes a PZT-8 crystal.

7. The apparatus of claim 1, wherein the ultrasound transducer includes a plurality of ultrasound transducers.

8. The apparatus of claim 1, further comprising:
an infrared camera disposed within a lumen defined by the elongate body configured to provide an indication of the temperature of the portion of the eye.

9. An apparatus, comprising:
an elongate body having a proximal end and a distal end and defining a lumen couplable to a source of suction; and
an ultrasound transducer coupled to the distal end of the elongate body,
the ultrasound transducer configured to contact an outer surface of an eye such that ultrasound energy can be applied to a targeted portion of the eye.

10. The apparatus of claim 9, further comprising:
a cooling member coupled to a proximal end of the ultrasound transducer.

11. The apparatus of claim 9, wherein the ultrasound transducer is an annular ring disposed around the lumen of the elongate body.

12. The apparatus of claim 9, wherein the ultrasound transducer includes a plurality of ultrasound transducers.

13. The apparatus of claim 9, wherein the ultrasound transducer includes a PZT-8 crystal.

14. The apparatus of claim 9, wherein the ultrasound transducer includes a piezo-electric film.

15. The apparatus of claim 9, wherein the elongate body is configured to provide suction through the lumen to the surface of the eye to stabilize the position of the elongate body.

16. A method, comprising:
disposing a medical device such that a distal end of the medical device is in contact with an outer surface of an eye, the medical device including a body and an ultrasound transducer coupled to a distal end of the body; and
applying ultrasound energy to a targeted portion of the eye while the medical device is disposed in contact with the outer surface of the eye to reshape the cornea of the eye.

17. The method of claim 16, further comprising:
applying suction through a lumen defined by the body while the medical device is in contact with the outer surface of the eye to stabilize the position of the medical device

18. The method of claim 16, further comprising:
monitoring a temperature of the targeted portion of the eye with an infrared camera disposed within a lumen of the body, during the applying ultrasound energy.

19. The method of claim 16, further comprising:
cooling the ultrasound transducer with a cooling member coupled to the ultrasound transducer during the applying ultrasound energy.

20. The method of claim 16, wherein the reshaping the cornea includes increasing the curvature of the cornea.

21. The method of claim 16, wherein the reshaping the cornea includes decreasing the curvature of the cornea.

22. The method of claim 16, further comprising:
applying suction through a lumen defined by the body while the medical device is in contact with the outer surface of the eye during the delivery of ultrasound energy to assist in reshaping the cornea of the eye.
